(19) United States
(12) Patent Application Publication
(10) Pub. No.: US 2016/0235802 A1
(43) Pub. Date: Aug. 18, 2016

(54) Traditional Chinese Medicine capable of triggering Lung Adenocarcinoma cell Autophagy, and composition and extract thereof

(71) Applicant: Kuan-chih CHOW, Taiwan (CN)

(72) Inventor: Kuan-Chih Chow, Nantou County (TW)

(21) Appl. No.: 14/903,277

(22) PCT Filed: Aug. 29, 2013
(86) PCT No.: PCT/CN2013/082592

§ 371 Date: Apr. 18, 2016

(51) Int. Cl. A61K 36/69; A61K 9/16; A61K 36/258; A61K 36/076; A61K 36/481; A61K 36/484; A61K 36/725; A61K 36/185; A61K 36/232; A61K 36/9068; A61K 36/906; A61K 9/00; A61K 36/284

(52) U.S. Cl. CPC A61K 36/69; A61K 9/0053; A61K 9/16; A61K 36/258; A61K 36/076; A61K 36/481; A61K 36/284; A61K 36/725; A61K 36/185; A61K 36/232; A61K 36/9068; A61K 36/906; A61K 36/484; A61K 2236/331; A61K 2236/33; A61K 2236/53; A61K 2236/37

(57) ABSTRACT

A Chinese medicine capable of triggering lung adenocarcinoma cell autophagy mainly uses a polygala tenuifolia willd herb with a quantity depending on a patient's physical fitness and sickness conditions determined by a doctor, and capable of triggering a lung adenocarcionma cell autophagy (cracking) to achieve a treatment effect. Regardless of being taken as a powder or an extract, the medicine is capable of triggering the adenocarcionma cell autophagy (cracking) effect to achieve the treatment effect. The polygala tenuifolia willd applies a new protein transport mode as a target enzyme screening to successfully prove the effect of triggering lung adenocarcionma cell autophagy (cracking) for cancer treatment.

### FIELD OF THE INVENTION

The technical field relates to a Chinese medicine capable of triggering lung adenocarcinoma cell autophagy, and more particularly to the Chinese medicine and its composition and extract capable of triggering lung adenocarcinoma cells to provide the effects of autophagy and cracking for cancer treatment.

### BACKGROUND OF THE INVENTION

Cancer is one of the top ten causes of death and has become the leading cause of death for 27 consecutive years. The main factor causing cancer is that cells become abnormal and keep dividing to form more abnormal cells automatically.

At present, Western medicine therapy such as surgery, radiation therapy, chemotherapy, hormone therapy, or biopharmaceutical therapy used for cancer treatment often causes serious side effects on a patient's body. Therefore, it is a blessing to cancer patients to have a mild treatment capable of triggering lung adenocarcinoma cell autophagy for cancer treatment.

Autophagy is a method for cells to maintain homeostasis and keep a high retentivity in eukaryote. When the cells are under stress, chemotherapeutic drug, TNFα, 1.25-dihydroxvitamin D, endotoxin, gamma interferon, radiation, high temperature, microbal infection, hypoxia, hunger, endoplasmic reticulum (ER) stress or mitochondria damage will induce and help the cell to adapt environmental changes. Autophagy is first found in vivo in yeast, and 27 types of directly participating specific genes are identified up to now, and these genes have a universal nomenclature of autophagy-related genes, atg), and there are also more than 50 other types of genes are related to autophagy, but the actual causal relation is still not clear. When the autophagy occurs, the cells will produce autophagosome with bilayer membranes, and these bilayer membranes are supplied by cell membrane, endoplasmic reticulum (ER), Golgi or mitochondrial outer membrane, but the actual mechanism is still a topic of discussion. The organelles and proteins covered by the bilayer membrane will form autolysosomes by the fusion of autophagosome and lysosome, and the enzyme in the lysosome digests and decomposes the substances inside the autolysosomes and recycles and reuses the substances.

Cell autophagy is mainly divided into four major types: macroautophagy, microautophagy, chaperone-mediated autophagy (CMA) and mitophagy, wherein macroautophagy is a major cell autophagy for proteins and organelles of greater phagocytosis; microautophagy is a cell autophagy for proteins of smaller phagocytosis, and the outer membrane of lysosome covers the protein to be degraded in cytoplasm; CMA carries target proteins into lysosome by chaperone to perform degradation; mitochondrial autophagy covers the mitochondria by autophagosome first, and then degrades the mitochondria. Mitochondrial autophagy is often considered as a part of macroautophagy and not included in the individual types of the cell autophagy. At present, three major mechanisms for controlling the function of the cell autophagy are known.

One of them is the ATPase family AAA domain containing 3A (ATAD3A) which is adenosine triphosphatase having a molecular weight of 67-kDa (kilodaltons) and existing in endoplasmic reticulum, mitochondria-associated membrane (MAM), transport vesicle, and mitochondrial outer membrane and it is a member of ATAD3 protein. Our study shows that the ATAD3A has the function of controlling the movement of the transport vesicle in cytoplasm. Clinically, a large amount of ATAD3A expresses in tissues of lung adenocarcinoma. The suppression of gene expression improves the apoptosis and autophagy of cancer cells as well as its sensitivity to chemotherapy drugs and radiation, and it shows that ATAD3A is an anti-apoptotic factor. In prostate cancer and cervical cancer, ATAD3A has the anti-autophagy capability. Silencing ATAD3A genes also suppresses the expression of mitochondria related proteins such as optical atrophy 1 (OPA1), the apoptosis initiation factor, and caspase 14, dynamin-related protein 1 (DRP1). When the ATAD3A antibody is used for the immunoprecipitation of light membrane and mitochondria-associated membrane (MAM) with non-denatured cytoplasm, AIF, caspase 14, OPA1, DRP land mitofusin 2 (Mfn-2) are also precipitated as shown in FIG. 1, and the immunoprecipitation and the two-dimensional silver stain indicate that the ATAD3A monoclonal antibody can precipitate Mfn-2, OPA1, DRP 1, eEF2 and AIF in the light membrane and the mitochondria-associated membrane (MAM) separated by the glucose gradient ultracentrifugation. The silenced DRP 1 causes a gigantic bubble-like structure in the cytoplasm, and the size of the bubble-like structure is 50 to 100 times of the mitochondria and such bubble-like structure aggregates a large quantity of cytochromes c (cyt c) and AIF. The observation and result of the aforementioned experiments allow use to assume that our discovery may be a new path for transporting protein to mitochondria, and such new path may not be the same as the conventional Pfanner model that transports the protein to the mitochondria by the cytoplasm directly.

Since the two proteins (AIF and cyt c) causes apoptosis in cytoplasm, therefore it is necessary to isolate the apoptosis related factors in the manufacture of proteins immediately. For example, phospholipids are used for the isolation. Therefore, we adopt AIF and cyt c proteins as the experiment model and assume that the new path of transporting protein to mitochondria may have the following mechanism and procedure. When a new protein is manufactured in the endoplasmic reticulum (ER), the former structure of the transport vesicle formed on the mitochondria-associated membrane (MAM) by the mitochondria-associated membrane (MAM) is budded off to form the transport vesicle which is fused into the mitochondria. This path requires three types of proteins, respectively: DRP1, ATAD3A and Mfn-2 and does not require translocase of mitochondrial outer membrane or inner membrane, and the translocase of the outer membrane is called TOM, and the translocase of the inner membrane is called TIM. With reference to FIG. 2 for the schematic view of the possible mechanism and procedure of transporting protein to mitochondria, when a new protein is manufactured in endoplasmic reticulum (ER) by mitochondria-associated membrane and transport vesicle and then fused into mitochondria, wherein this path requires the three proteins, respectively: DRP1, ATAD3A and Mfn-2, so that the silenced individual genes may affect the special structure of different organelles in the cytoplasm, and silencing these genes will affect the special structure of organelles in the cytoplasm and reduce the protein content of the mitochondria as shown in FIG. 3, and our assumed path of transporting protein to mitochondria requires three types of proteins: DRP 1, ATAD3A and Mfn-2, so that the individual silenced genes will affect the special structure of organelles in cytoplasm. The expression of silenced ATAD3A (the middle row) reduces the overlapping (yellow fluorescent light, the third and fourth columns) of the endoplasmic reticulum (ER, KDEL-combined with green fluorescent light, the first column) and the mitochondria (red fluorescent light, the second column). The expression of ATAD3A also reduces the number of mitochondria but increases the endoplasmic reticulum (ER, the middle row, the fourth column, white arrow). The expression of the silenced DRP1 (DRP1kd) increases the gigantic endoplasmic reticulum (ER, the third row, the fourth column, white arrow) instead. Wherein, N represents cell nucleus. In FIG. 4, the formation of ATAD3A and Mfn-2 genes in the transport vesicle and the transportation process play different roles respectively, so that the silencing of individual genes will produce bubble-like structures in different forms in the cells.

It is noteworthy that mitochondria does not manufacture phosphatidylserine (PS) required by the organelle membrane, and PS is formed by endoplasmic reticulum (ER) and of PS synthase 1 (PSS1, CDP-diacylglycerol:L-serine O-phosphatidyltransferase) of MAM to be converted into (phosphatidylcholine, PC) and then supplied to the mitochondria. An intramembranous space of the mitochondria is situated at PS decaroxylase 1 (PSD1) on the inner membrane and capable of converting the PS into phosphatidylethanolamine (PE) and then flowing back to the endoplasmic reticulum (ER) and other organelles. However, the mechanism of transporting the phospholipid between the endoplasmic reticulum (ER) and the mitochondria is just explained in today's journals with regard to a phospholipids exchange mechanism. Since using the mechanism of the phospholipids exchange headgroup (such as choline, ethanolamine, serine, etc) occurred on the same organelle membrane to explain the transportation or exchange of phospholipids between two different types of organelle membranes is inappropriate, or even using a method similar to CERT also has a certain limitation. Our newly found path of transporting proteins not just explains how the protein is transported from the endoplasmic reticulum (ER) to the mitochondria only, but also explains how the phosphatidylserine (PS) and protein are transported by the endoplasmic reticulum (ER) to the mitochondria at the same time.

Recently, we found out that the protein sequence of the ATAD3A is very similar to the protein sequence of the vacuolar protein sorting (VPS)4 related to the induction of the atg8-PE-independent formation of multivesicular bodies (MVB) in the ATP enzyme functional motifs of small bubbles in the moving cytoplasm. Since ATAD3A is an ATP enzyme for moving the transport vesicle and both ATAD3A and VPS4 may have similar functions. In the silenced cells of the ATAD3A genes, mitochondria may produce a large quantity of PE when the supply of mitochondria protein and PS is insufficient. As a result, an autophagy is induced naturally to maintain the function of balancing the organelles.

According to our new path of transporting mitochondria proteins, we understand that the metabolism and transportation of tissues from cell membranes to cell nuclei of the continuous large membrane tissue other than the continuous membrane tissue, which includes mitochondria, Golgi, endosome, lysosome, autophagosome, protein between organelles, phosphate ester, and sphingomyelin should be synchronous and may be related to the endoplasmic reticulum (ER), mitochondria-associated membrane (MAM) and transport vesicle, and the movement of the transport vesicle and the fusion between non-continued large membrane tissues require the proteins including DRP1, ATAD3A and Mfn-2, and the connection of these intracellular membrane tissues relates to viral infection and damage to organelle structure which are advantages to the mechanism of reproducing viruses, and the expression of innate immune response and adaptive immune response and antigen, and understanding such information is the basic for development new treatment methods.

In addition, it is noteworthy that shikonin is capable of inhibiting HIV replication and also inducing cell autophagy. Particularly, at an early drug treatment (for a short time), the cell morphology is similar to that of DRP lkd and has a fat ER/MAM structure, implying that the efficacy of shikonin affects the functions of DRP1 and ATAD3A, so as to induce the cell autophagy.

Our job is to understand the autophagy and the relation between the cellular detoxification and the drug resistance in order to learn the main function of ATAD3A, hHR23A, LC3-II and ceramide on the formation of cell autophagosomes.

### SUMMARY OF THE INVENTION

At present, we have applied a new protein transport mode as a target enzyme screening Chinese medicine and successfully screened polygala tenuifolia willd in Chinese medicine having such effect. Other Chinese medicine are ground into powder according to the medicine property of the polygala tenuifolia willd to produce a composition of this disclosure, and different formulae and parts by weight are used for extraction to produce an extract of this disclosure.

Therefore, it is a primary objective of this disclosure to provide a Chinese medicine capable of triggering lung adenocarcinoma cell autophagy and its composition and extract which can be taken orally by a patient to achieve the effects of producing autophagy or cracking of lung adenocarcinoma cells and treating cancers.

The Chinese medicine capable of triggering lung adenocarcinoma cell autophagy in accordance with this disclosure comprising a polygala tenuifolia willd herb with a quantity depending on a patient's physical fitness and sickness conditions determined by a doctor, and capable of triggering a lung adenocarcionma cell autophagy (cracking) to achieve a treatment effect.

Wherein, the polygala tenuifolia willd herb is prepared in form of a powder to be taken orally by a patient.

Wherein, the polygala tenuifolia willd herb is taken with 5 g of a water-quenched medicine soup obtained from a filtered pharmaceutical residue after a water quenching procedure, a liquor-quenched medicine soup obtained from a filtered pharmaceutical residue after a liquor quenching procedure, and the water-quenched medicine soup and the liquor-quenched medicine soup are mixed and taken orally by a patient.

Wherein, the water quenching procedure heats the herbs by 100 g of water at 60° C. for 20 minutes, and the liquor quenching procedure heats the herbs by 100 g of liquor with an alcohol content of 50˜90% at 60° C. for 15 minutes .

The Chinese medicine composition capable of triggering lung adenocarcinoma cell autophagy in accordance with this disclosure comprises a plurality of herbs including 1 part by weight of polygala tenuifolia willd, 2 parts by weight of ginseng, 2 parts by weight of poria, 2 parts by weight of astragalus membranaceus, 2 parts by weight of atractylodes macrocephala, 2 parts by weight of logan meat, 2 parts by weight of ziziphus jujaba mill var.spinosa, 1 part by weight of angelica sinensis, 1 part by weight of ziziphus jujaba, 1 part by weight of fresh ginger 0.5 part by weight of costustoot, and 0.5 part by weight of licorice, grounded into powder to be taken orally by a patient.

The Chinese medicine extract capable of triggering lung adenocarcinoma cell autophagy in accordance with this disclosure comprises 6 parts by weight of polygala tenuifolia willd added with a plurality of herbs including 6 parts by weight of ginseng, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

The Chinese medicine extract capable of triggering lung adenocarcinoma cell autophagy in accordance with a second preferred embodiment of this disclosure comprises 6 parts by weight of polygala tenuifolia willd added with a plurality of herbs including 6 parts by weight of codonopsis pilosula, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger herbs, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

The Chinese medicine extract capable of triggering lung adenocarcinoma cell autophagy in accordance with a third preferred embodiment of this disclosure comprises 6 parts by weight of polygala tenuifolia willd, added with a plurality of herbs including 6 parts by weight of American ginseng, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

Wherein, the water quenching procedure heats the herbs by 100 g of water at 60° C. for 20 minutes; and the liquor quenching procedure heats the herbs by 100 g of alcohol with an alcohol content of 50˜90% at 60° C. for 15 minutes.

### BRIEF DESCRIPTION OF THE DRAWINGS

FIG. 1 is a schematic view of immunoprecipitation and two-dimensional electrophoresis showing that Mfn-2, OPA1, DRP1, eEF2 and AIF may be precipitated from a light membrane and a mitochondria-associated membrane (MAM) separated by glucose gradient ultracentrifugation by monoclonal antibodies of ATAD3A;

FIG. 2 is a schematic view showing possible mechanism and procedure of a new path of transporting protein to mitochondria;

FIG. 3 is a schematic view of a special structure of organelles in cytoplasm which is affected by individual silenced genes;

FIG. 4 is a schematic view of different bubble-like structures formed in cells during the formation and transportation process of ATAD3A and Mfn-2 genes in the transport vesicle;

FIG. 5 shows computer tomography scans comparing the conditions of a lung adenocarcinoma patient after having the treatment of this disclosure for eight weeks and before having the treatment; and

FIG. 6 shows cross-sectional computer tomography scans comparing the conditions of a lung adenocarcinoma patient after having the treatment of this disclosure for eight weeks and before having the treatment.

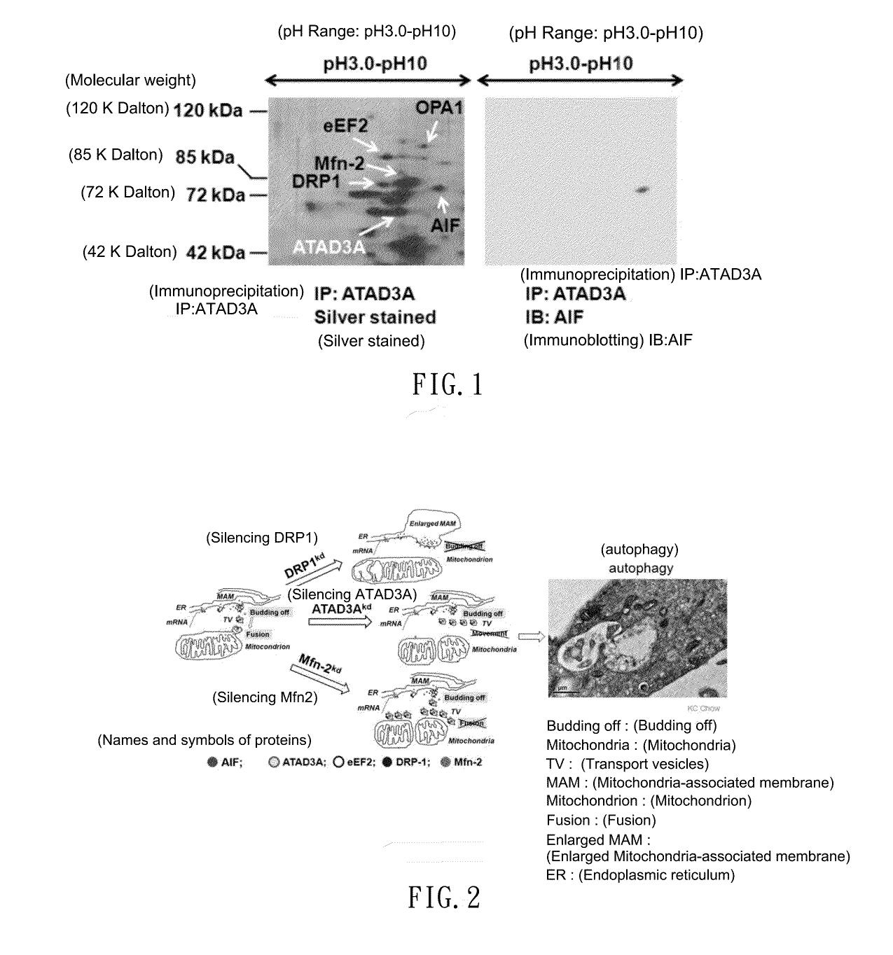

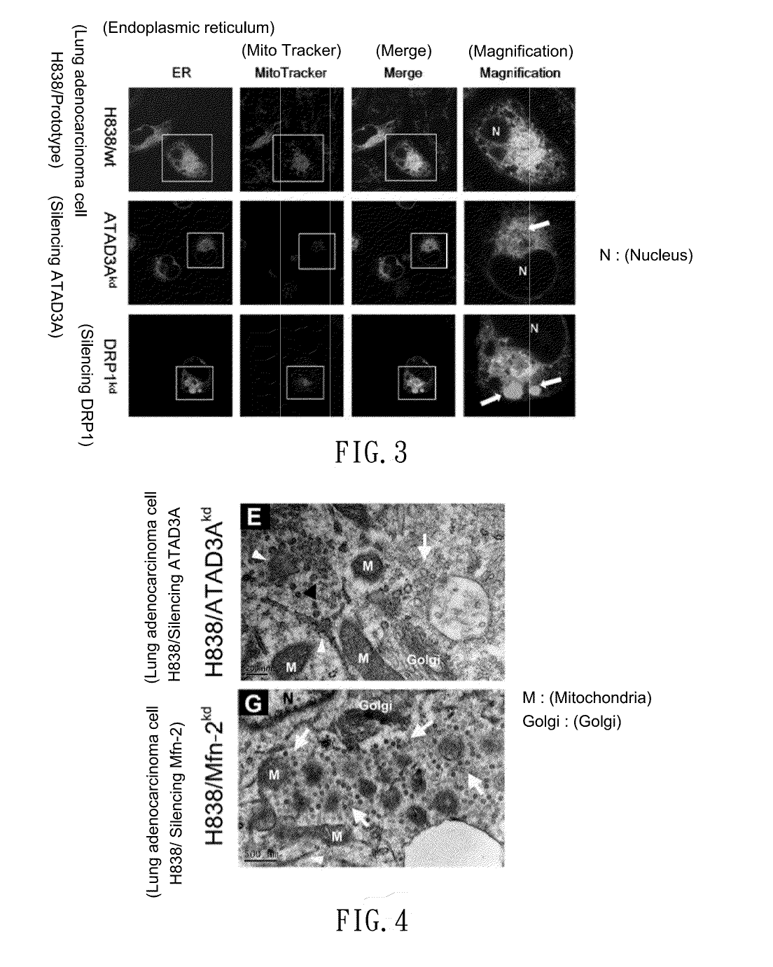

### DESCRIPTION OF THE PREFERRED EMBODIMENTS

The aforementioned and other objectives and advantages of this disclosure will become clearer in light of the following detailed description of an illustrative embodiment of this invention described in connection with the drawings. The embodiment briefly describes the cause of triggering the cell autophagy and related experiment data for supporting the advantages and effects of this disclosure.

The adenocarcinoma cell autophagy mainly uses polygala tenuifolia willd herb with a dosage depending on a patient's physical fitness and sickness condition determined by a doctor. The medicine is taken orally by a patient, regardless of its form of powder or extract.

The polygala tenuifolia willd of this disclosure is a perennial herb or a root of Polygala tenuifolia willd sibirica L. mainly produced in Hebei, Shanxi, Shaanxi, Jilin, Henan, and other places. After a pre-emergence in spring or a partial withered on ground in autumn, the root is dug, and the residue and mud are removed before drying. The polygala tenuifolia willd may be taken raw or cooked and has the effects of calming, expectorant resuscitation, and eliminating carbuncles.

In general, polygala tenuifolia willd is primarily used to achieve the following four effects: 1. It is used for improving palpitations, insomnia, and forgetfulness. Polygala tenuifolia willd penetrates into heart and kidney, and is thus capable of opening up a patient's mind to clamp the patient and communicating with kidney to strength the patient's memory, which is an excellent herb for the communication of heart and kidney and setting a patient to a clam and peaceful state. Therefore, this herb is usually used for curing restlessness, palpitation, insomnia, and forgetfulness, and it is compatible with ginseng, dragon teeth herb, and poria such as the sedative and heart invigorating pill. 2. It is used for improving the conditions of phlegmatic mental confusion, epilepsy, and madness. This herb tastes bitter and flat and is capable of suppressing phlegm, and calming excited mental condition, so that it can be used for curing epileptic convulsion and phlegmatic madness caused by phlegmatic mental confusion. This herb may be used together with pinellia, tianma, and scorpion for curing epileptic faintness and spastic convulsion and with acorns tatarinowii, curcuma aromatica, and alumen for curing madness and insanity. 3. This herb may be used for curing cough and phlegm. This herb penetrates into a patient's lung to eliminating phlegm and preventing cough, and thus is particularly good for patients with thick viscose or sputum or unable to cough or spit smoothly. This herb is generally used together with almond, fritillaria, and platycodon, etc. 4. This herb is used for curing ulcer and sore, and swelling and pain of breast. This herb tastes bitter, warm and smooth, and is thus good for clearing the jam of blood circulation and removing swellings and capable of curing various different ulcers. Regardless of the patient's cold, hot, weak or strong conditions, this herb is used solely in form of powder and taken together with rice wine, or applied externally to a patient's affected area to achieve the aforementioned effects.

This disclosure applying the new protein transport mode as the target enzyme to screen the polygala tenuifolia willd further has the effect of triggering adenocarcinoma cell autophagy. As indicated by the middle arrow of FIG. 2, the mitochondria in the silenced cells of the ATAD3A genes produces a large quantity of PE when there is insufficient mitochondria protein and resupply of PS, so that the autophagy is induced naturally, and the polygala tenuifolia willd contains an ingredient that affects the function of the ATAD3A, so as to induce the cracking of adenocarcinoma cells and achieve the effect of cancer treatment.

The Chinese medicine “polygala tenuifolia willd” screened by this disclosure can be taken orally in form of powder (a scientific Chinese medicine) or an extract to achieve the effect of triggering adenocarcionma cell autophagy (or cracking), so as to achieve the treatment effect.

When the medicine is orally taken in form of an extract, 5 g of polygala tenuifolia willd is prepared, and processed by a water quenching procedure (that heats the herbs by 100 g of water at 60° C. for 20 minutes to filter a pharmaceutical residue and obtain a water-quenched medicine soup, and the filtered pharmaceutical residue is further processed by a liquor quenching procedure (that heats the herbs by 100 g of alcohol with an alcohol content of 50˜90% at 60° C. for 15 minutes) to filter a pharmaceutical residue and obtain a liquor-quenched medicine soup; and the water-quenched medicine soup and the liquor-quenched medicine soup are mixed and then orally taken by a patient.

This disclosure is applied to EGFR mutation-negative lung cancer patients through the cooperation with Chinese medicine doctor to achieve a very good effect.

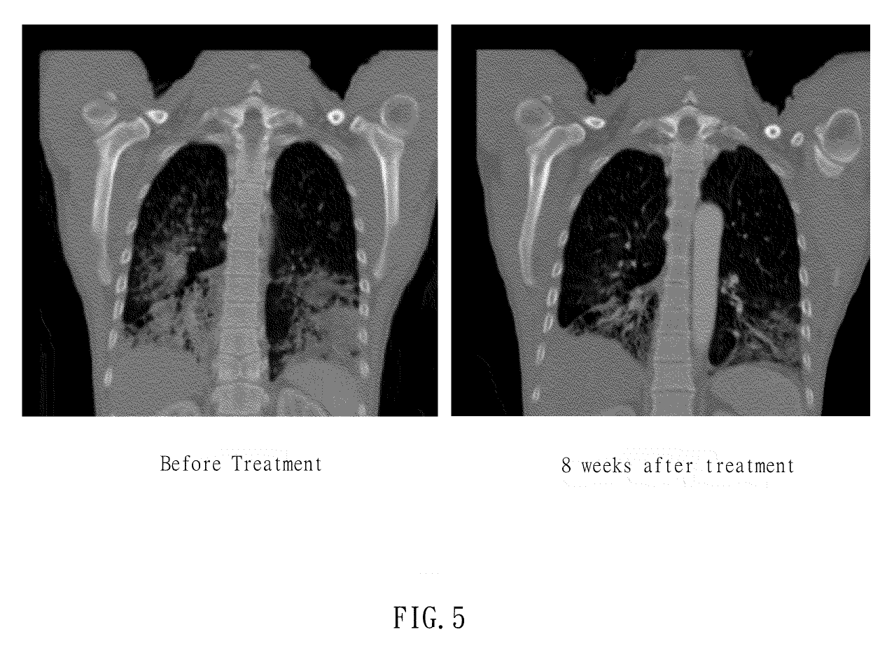

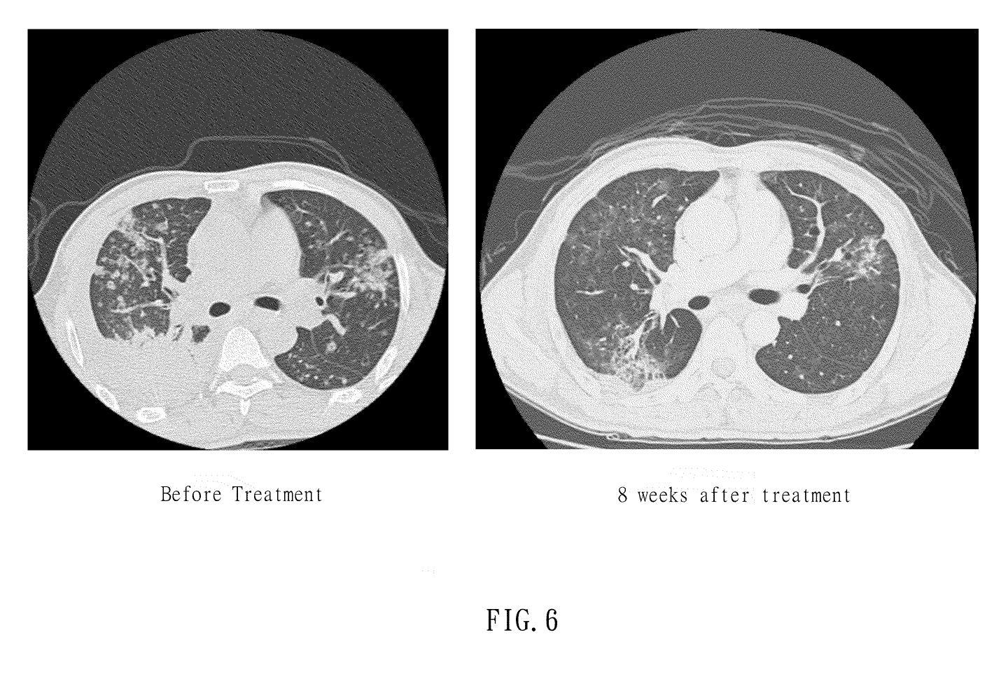

With reference to FIGS. 5 and 6 for computer tomography scans and cross-sectional computer tomography scans comparing the conditions of a lung adenocarcinoma patient after having the treatment of this disclosure for eight weeks and before having the treatment respectively, the white cancerous tumor lesion area of the patient's lung is reduced significantly after taking the Chinese medicine of this disclosure after eight weeks of treatment, and it shows that the Chinese medicine of this disclosure definitely has the effects of triggering lung adenocarcinoma cell autophagy and curing cancer.

In addition, the Chinese medicine composition capable of triggering lung adenocarcinoma cell autophagy in accordance with this disclosure is formed by combining other Chinese medicine according to the medicine nature of the polygala tenuifolia willd, and such composition comprises 1 part of polygala tenuifolia willd, added with a plurality of herbs including 2 parts by weight of ginseng, 2 parts by weight of poria, 2 parts by weight of astragalus membranaceus, 2 parts by weight of atractylodes macrocephala, 2 parts by weight of logan meat, 2 parts by weight of ziziphus jujaba mill.var.spinosa, 1 part by weight of angelica sinensis, 1 part by weight of ziziphus jujaba, 1 part by weight of fresh ginger, 0.5 part by weight of costustoot, and 0.5 part by weight of licorice, appropriately ground into powder to be orally taken by a patient.

The Chinese medicine extract capable of triggering lung adenocarcinoma cell autophagy in accordance with a first preferred embodiment of this disclosure comprises 6 parts by weight of polygala tenuifolia willd added with a plurality of herbs including 6 parts by weight of ginseng, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

In addition, the Chinese medicine extract capable of triggering lung adenocarcinoma cell autophagy in accordance with a second preferred embodiment of this disclosure comprises 6 parts by weight of polygala tenuifolia willd added with a plurality of herbs including 6 parts by weight of codonopsis pilosula, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger herbs, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

Finally, the Chinese medicine extract capable of triggering lung adenocarcinoma cell autophagy in accordance with a third preferred embodiment of this disclosure comprises 6 parts by weight of polygala tenuifolia willd, added with a plurality of herbs including 6 parts by weight of American ginseng, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

The water quenching procedure heats the herbs by 100 g of water at 60° C. for 20 minutes; and the liquor quenching procedure heats the herbs by 100 g of alcohol with an alcohol content of 50˜90% at 60° C. for 15 minutes.

In addition, the Chinese medicine capable of triggering lung adenocarcinoma cell autophagy and its composition and extract in accordance with this disclosure may increase or decrease the proportion of the parts by weigh of the polygala tenuifolia willd to adjust the effect of cancer cell autophagy.

The Chinese medicine capable of triggering lung adenocarcinoma cell autophagy and its composition and extract in accordance with this disclosure have the effect of triggering lung adenocarcinoma cell autophagy (or cracking) to result in the self apoptosis of the lung adenocarcinoma cells, so as to achieve the cancer treatment effect.

In summation of the description above, this disclosure is novel, inventive, useful, and in compliance with patent application requirement, and thus is filed for patent application. While this disclosure has been described by means of specific embodiments, numerous modifications and variations could be made thereto by those skilled in the art without departing from the scope and spirit of this disclosure set forth in the claims.

What is claimed is:

1. A Chinese medicine capable of triggering lung adenocarcinoma cell autophagy, comprising a polygala tenuifolia willd herb with a quantity depending on a patient's physical fitness and sickness conditions determined by a doctor, and capable of triggering a lung adenocarcionma cell autophagy (cracking) to achieve a treatment effect.

2. The Chinese medicine capable of triggering lung adenocarcinoma cell autophagy according to claim 1, wherein the polygala tenuifolia willd herb is prepared in form of a powder to be taken orally by a patient.

3. The Chinese medicine capable of triggering lung adenocarcinoma cell autophagy according to claim 1, wherein the polygala tenuifolia willd herb is taken with 5 g of a water-quenched medicine soup obtained from a filtered pharmaceutical residue after a water quenching procedure, a liquor-quenched medicine soup obtained from a filtered pharmaceutical residue after a liquor quenching procedure, and the water-quenched medicine soup and the liquor-quenched medicine soup are mixed and taken orally by a patient.

4. The Chinese medicine capable of triggering lung adenocarcinoma cell autophagy according to claim 1, wherein the water quenching procedure heats the herbs by 100 g of water at 60° C. for 20 minutes, and the liquor quenching procedure heats the herbs by 100 g of liquor with an alcohol content of 50˜90% at 60° C. for 15 minutes.

5. A Chinese medicine composition capable of triggering lung adenocarcinoma cell autophagy, comprising a plurality of herbs including 1 part by weight of polygala tenuifolia willd, 2 parts by weight of ginseng, 2 parts by weight of poria, 2 parts by weight of astragalus membranaceus, 2 parts by weight of atractylodes macrocephala, 2 parts by weight of logan meat, 2 parts by weight of ziziphus jujaba mill var.spinosa, 1 part by weight of angelica sinensis, 1 part by weight of ziziphus jujaba, 1 part by weight of fresh ginger 0.5 part by weight of costustoot, and 0.5 part by weight of licorice, grounded into powder to be taken orally by a patient.

6. A Chinese medicine extract capable of triggering lung adenocarcinoma cell autophagy, comprising 6 parts by weight of polygala tenuifolia willd added with a plurality of herbs including 6 parts by weight of ginseng, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill.var.spinosa, 3 parts by weight of ziziphus jujaba, 1.5 parts by weight of radix glycyrrhizae preparata, 3 parts by weight of logan meat, and 1.5 parts by weight of ginger, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

7. A Chinese medicine extract capable of triggering lung adenocarcinoma cell autophagy, comprising 6 parts by weight of polygala tenuifolia willd added with a plurality of herbs including 6 parts by weight of codonopsis pilosula, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger herbs, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

8. A Chinese medicine extract capable of triggering lung adenocarcinoma cell autophagy, comprising 6 parts by weight of polygala tenuifolia willd, added with a plurality of herbs including 6 parts by weight of American ginseng, 3 parts by weight of atractylodes macrocephala, 3 parts by weight of poria pararadicis, 3 parts by weight of astragalus membranaceus, 3 parts by weight of costustoot, 3 parts by weight of angelica sinensis, 3 parts by weight of ziziphus jujaba mill var.spinosa, 3 parts by weight of ziziphus jujaba, 1.5 parts by weight of radix glycyrrhizae preparata, 3 parts by weight of logan meat, and 1.5 parts by weight of ginger, and having a total weight of 5 g; a pharmaceutical residue being filtered to produce a water-quenched medicine soup in a water quenching procedure; a pharmaceutical residue being filtered to produce a liquor-quenched medicine soup in a liquor quenching procedure of the previously filtered pharmaceutical residue; and the water-quenched medicine soup and the liquor-quenched medicine soup being mixed to form a Chinese medicine extract.

9. The Chinese medicine extract capable of triggering lung adenocarcinoma cell autophagy according to any one of claims 6, 7 and 8, wherein the water quenching procedure heats the herbs by 100 g of water at 60° C. for 20 minutes; and the liquor quenching procedure heats the herbs by 100 g of alcohol with an alcohol content of 50˜90% at 60° C. for 15 minutes.
